(19) United States
(12) Patent Application Publication
(10) Pub. No.: US 2006/0062733 A1
(43) Pub. Date: Mar. 23, 2006

(54) Medicament/dosimeter combination packaging

(75) Inventors: Hans Schreier, Neuss (DE); Wolfgang Greb, Dusseldorf (DE)

Correspondence Address: GUDRUN E. HUCKETT DRAUDT, LONSSTR. 53, WUPPERTAL, 42289, DE

(73) Assignee: MCS Micro Carrier Systems GmbH, Neuss (DE)

(21) Appl. No.: 10/528,783

(22) PCT Filed: Sep. 24, 2003
(86) PCT No.: PCT/DE03/03186

§ 371 Date: Apr. 21, 2005

(30) Foreign Application Priority Data
Sep. 27, 2002 (DE) 102 45 508.2

(51) Int. Cl. A61K 49/00

(52) U.S. Cl. 424/10.1

(57) ABSTRACT

In a system for individual dosing of a medicament, in accordance with the individual pathological properties (“fingerprint”) of a patient—corresponding physiologically or genetically to the disease status—two components are provided: a medicament that is to be taken or administered in a variable individual dosage and a miniaturized indicator system that obtains information from blood, saliva or other bodily fluids and tissues of the patient and displays the information in a readable form so that the patient or the treating physician can immediately read the optimal active substance to be taken or dosage to be administered.

The present invention concerns a system for individual dosing of a medicament, in accordance with the individual pathological properties (“fingerprint”) of a patient—corresponding physiologically or genetically to the disease status. The system is comprised of two components: the medicament that is to be taken or administered in a variable individual dosage and a miniaturized indicator system that obtains information from blood, saliva or other bodily fluids and tissues of the patient and displays the information in a readable form so that the patient or the treating physician can immediately read the optimal dosage to be taken or to be administered.

The human genome research and the resulting identification of a large number of genes (active locations) has also revolutionized the field of diagnostic medicine. The genetic foundations of many processes occurring within the body, for example, metabolic processes controlled by enzymes, are already known. Genes regulate also the activity of cellular enzymes, that inter alia individually determine the metabolic conversion, resorption, and action or side effects of medicaments. Moreover, more and more genetic mutations and the resulting defects or diseases can be detected with relatively quick and precise methods. This information in regard to individual characteristics of cell activity of a patient is employed in the field of medical treatment for the optimal application of an appropriate medicament with regard to type, dosage and dosage intervals of an appropriate medicament.

The object of the present invention is a medicament/dosimeter combination package comprising in a packaging:
a) a medicament that can be individually dosed, and,
b) a diagnostic indicator system for an endogenous substance, regulation mechanism, or gene, or indication system, relevant for the action, side effect, interaction, metabolism, absorption, distribution, metabolism, and elimination of the medicament to be administered.

The system according to the invention is suitable in particular for the individual dosing of a medicament, in accordance with the individual pathological and physiological or genetic properties (“fingerprint”) of the patient. This system is comprised of two components: the medicament that is to be taken or administered in variable individual dosage and a miniaturized indicator system that obtains information derived from blood, saliva or other bodily fluids and tissues of the patient and displays the information in readable form so that the patient or the physician treating the patient can read immediately the optimal dosage to be taken or to be administered. In special situations, it can also be derived from the information whether taking a certain medicament will even cause a therapeutic effect (responder/non-responder definition).

The use of the present invention is realized at three different levels:
1. as an analytical measuring unit before taking or dosing a medicament in order to define the (genetic) type of the patient and to derive therefrom a conclusion whether the patient is to be treated or not to be treated with a specific medicament or with a specific quantity of a medicament;
2. as a dosage metering unit during administration of the medicament in order to make available to the patient continuously the optimal dosage of the medicament;
3. as a monitoring measuring unit that continuously measures and documents the effect of a medicament and thus enables the patient and/or the physician to continuously monitor the success or failure of a medication.

While the medicament itself can be present in any pharmaceutical form, as a solution, drops, tablets, micro pellets, cream, inhalant etc., the indicator system in the simplest form will be comprised of a paper or plastic strip having at its end a reactive zone that is to be brought into contact with the bodily fluid. This can be either a paper strip that is impregnated with a reaction mixture or a depression (well) or a receptacle with indicator solution or reaction solution in which the bodily fluid and the chemical liquid are mixed. In the simplest case, the result of the reaction (signal) will be simply a color change and, as such, can indicate to the patient or the physician a range or an exclusion limit that can be used then by the patient or physician to decide on the amount of medication to be taken.

The test system can however be based also on a quantitative chemical reaction whose result cannot only be estimated simply by the naked eye but can be detected and read quantitatively as a defined value by inserting the test strip or the test well into an appropriate analytical device (“indicator”). The analytical device itself can be connected to a database having already stored therein historical data of the patient that can therefore be used further for making a decision.

The test system can also be comprised of a chip that is coated with one or several reactive substances and, after reaction with the applied bodily fluid, provides one or several measurable results and proposes or permits or excludes dosage quantities.

In an ideal situation, test system and medicament form are interconnected with one another such that, by combining the chip with an appropriate chip on or in the medicament form, the latter indicates or releases the optimal dosage of the medicament for the respective state based on the information that is available without any action to be preformed by the patient or physician. Examples for this are a cassette that releases in accordance with the read-out information a certain quantity of capsules or tablets; a programmed droplet dispenser or cream dispenser; a subcutaneous injection, for example, with a “pen injector” that injects subcutaneously an amount of medication that is precisely defined but variable depending on the individual information; or a variable atomizer that atomizes according to the information a quantity of a substance that is then to be inhaled by the patient.

Possible embodiments of the invention include the following combinations.

In the simplest case, the medicament/dosimeter combination package has two separate compartments; in one of them the medicament is stored in a certain administration form, and in the other one a corresponding number of test strips is stored. In this case, it is within the hands of the patient or the physician to use the indicator system before administering an appropriate dosage.

However, the medicament/dosimeter combination package can be constructed such that the patient or the physician first must remove, e.g., pull out or break off, the indicator strip before the medicament can be dispensed for administration.

In the ideal situation, the medicament/dosimeter combination package is constructed such that the patient or the physician must perform the test with the indicator system before the medicament can be released in a certain dosage based on the test. This can be realized best with an indicator system that operates on the basis of chips because in this way quantitative information can be transmitted directly onto a mechanical system so that, for example, a wheel containing pills and provided with a rotary mechanism can be opened only by a defined number of rotations corresponding to a certain dosage of the medicament contained in the tablets or capsules.

In one possible embodiment, the subject matter of the present invention is configured as a container filled with small tablets, pellets, or micro pellets that releases a defined amount/number of solid bodies. In another embodiment of the present invention, the container contains a liquid substance wherein a defined volume is released for oral, sublingual, or topical application, respectively.

A further example would be an injector that, based on the information transmitted by the chip injects only a certain volume of medicament, for example, subcutaneously, or an aerosol device that, based on the information of the chip inserted into the device, atomizes a certain volume containing a defined amount of medicament that is then inhaled by the patient.

In the ideal situation, the transmission is realized wireless and in real time (for example, Bluetooth technology).

### EXAMPLES

1. Antibody Therapy

Determination of a responder/non-responder situation on the basis of a certain gene expression and corresponding decision which therapy concept is optimal.

In the tumor tissue of a female patient, the expression of HER2 is measured before beginning a breast cancer therapy with antibodies against HER2. Only when HER2 is over-expressed, the therapy is employed.

2. Anti-Estrogen Therapy (for Example, with Tamoxifen) or Other Tumor Therapies

Optimization of the effect with simultaneous reduction of side effects as much as possible.

In certain time intervals, the female patient performs an estrogen receptor expression test with the indicator system that is made available. Based on the measured number of receptors, an individual dosage is calculated. Alternatively, a tumor marker is determined (for example, M2-PK, CEA, MUC-1, etc.) that indicates the suppression or spreading of metastases of the tumor. In this way, for the temporal course of a long-term treatment the optimal dosage is always made available.

3. Lipid Lowering (Statines)

Control or avoidance of side effects, or monitoring of the interaction potential in the case of multidrug therapy (alert system).

The creatine kinase (CK) or elastase derived from the serum of the patient is measured before taking the lipid lowering agent. The dosage is lowered or the medicament is discontinued when an increased rhabdomyolysis (enzymatic muscle breakdown) is calculated based on the measured concentration of CK or elastase.

4. Beta-Blocker (High Blood Pressure or Post-MI)

Adaptation of the dosage to a changed target expression so that the medicament efficacy is maintained at the same level.

A receptor expression test is performed so that a change of the number of receptors (up/down regulation) is detected and dosage is adjusted accordingly.

5. Antidepressive agents (or other Cyt. P450 inhibiting or stimulating medicaments).

Detection of gene expression or of a metabolic/enzymatic process that can occur at different rates (slow/fast) and that requires a corresponding adaptation of the dosage to the metabolic rate that is to be expected.

Depending on the genetic predisposition, the enzyme cytochrome P450 that is responsible for the metabolism can be inhibited or especially active upon taking antidepressive agents. This can lead to inhibition of the metabolism of the taken antidepressive agents but also of other medicaments. Accordingly, dangerously high plasma levels can result. On the other hand, a very active metabolism prevents the build-up of an effective blood level. Cytochrome P450 of a patient is characterized with a gene expression chip directly or indirectly by metabolic conversion of an appropriate substrate. Based on the result, the patient is classified as a slow/fast metabolizer and the dosage of the antidepressive agent is adjusted accordingly.

Based on the presented examples, it becomes clear that it is indeed conceivable to treat patients individually instead of according to a generalized treatment scheme that is based on statistic information but is too coarsely incremented. The consequence is an improved efficacy of the medicament that is adjusted individually wherein in the ideal situation also a significant reduction of side effects is observed because of the optimization of the dosage as well as of the dosage interval. This leads generally to a higher probability of curing as well as improved quality of life for the patient as well as to a reduction of the total costs and thus a positive economic effect for the patient or the health-care system.

In special situations, it is moreover also possible to shelter from the beginning so-called non-responders from ineffective (nonsensical) treatment that often causes severe side effects.

What is claimed is:

1.-7. (canceled)

8. A medicament/dosimeter combination package comprising:
a) a medicament to be individually dosed, and
b) a diagnostic indicator system for a patient-specific property that is relevant for the action, side effect, interaction, metabolism, absorption, distribution, metabolism, and elimination of the medicament to be administered to a patient, wherein the patient-specific property is selected from the group consisting of an endogenous substance, a regulation mechanism, a gene or an indication system.

9. The combination package according to claim 8, wherein the diagnostic indicator system is comprised of a strip impregnated with a chemical; a well that is coated with a chemical or biological indicator substance; or an electronic chip that is coated with a chemical or biological indicator substance.

10. The combination package according to claim 8, wherein the diagnostic indicator system provides information visually, spectrophotometrically, fluorometrically, or electronically.

11. The combination package according to claim 8, wherein individual dosing of the medicament is realized by a mechanical or electronic calibration of a tablet, a tablet container, a liquid, a liquid container, a dispenser, an injector, a tube for semi-solid medicaments, or a container for atomizing liquids or powders.

12. The combination package according to claim 8 providing an individually adjusted therapy for the patient in accordance with a physiological or pathological state of the patient.

13. The combination package according to claim 8 providing simple and unequivocal handling by the patient or nursing personnel or a physician.
